(19) United States
(12) Patent Application Publication
(10) Pub. No.: US 2023/0256634 A1
(43) Pub. Date: Aug. 17, 2023

(54) Method and Apparatus for a Clipper

(71) Applicant: Medline Industries, LP, Northfield, IL (US)

(72) Inventors: Stuart Mintz, Glenview, IL (US); Kaitlyn Farley, Chicago, IL (US); Robert Lockwood, Libertyville, IL (US)

(21) Appl. No.: 18/139,085

(22) Filed: Apr. 25, 2023

Related U.S. Application Data
(63) Continuation of application No. 16/848,186, filed on Apr. 14, 2020.
Application No. 16/848,186 is a continuation of application No. 15/260,623, filed on Sep. 9, 2016, now abandoned.
Application No. 15/260,623 is a continuation of application No. 14/193,367, filed on Feb. 28, 2014, now abandoned.

(51) Int. Cl. B26B 19/38; B26B 19/44; B26B 19/46; A61B 90/00

(52) U.S. Cl. CPC B26B 19/388; B26B 19/3873; B26B 19/386; B26B 19/3853; B26B 19/44; B26B 19/46; A61B 90/08

(57) ABSTRACT

An electric clipper having a body, a motor disposed within the body, a clipper head that operably couples to the body and that is powered by the motor, a power actuator configured to power on and off the motor such that when the motor is powered on by the power actuator the motor operates at a normal level of performance and at least one boost actuator configured to provide increased operating performance of the clipper head when actuated by a user. By one approach the increased operating performance is provided for only a set amount of time.

### CROSS-REFERENCE TO RELATED APPLICATIONS

This application is a continuation of U.S. patent application Ser. No. 16/848,186, filed Apr. 14, 2020, which is a continuation of U.S. patent application Ser. No. 15/260,623, filed Sep. 9, 2016, which is a continuation of U.S. patent application Ser. No. 14/193,367 filed Feb. 28, 2014, each of which are incorporated herein by reference in their entireties.

### TECHNICAL FIELD

These teachings relate generally to clippers.

### BACKGROUND

Clippers, shears, or electric razors, and more particularly medical clippers, are often used in preparation of an area of a patient for surgical procedures. Often, clippers utilize a rechargeable power source, such as one or more rechargeable batteries encapsulated within the housing of the clipper. Accordingly, the clippers must typically be recharged often to ensure enough power is available for future uses. Typically, recharging involves physically connecting the clippers to a power source (such as mains power from a wall outlet, or the like) or physically placing the clippers in a charging station.

Though suitable for at least some purposes, such an approach does not necessarily meet all needs of all application settings and/or all users. For example, users can fail to replace clippers onto a charging station or connect them to a power source after use, thus potentially leading to unexpected power loss during use due to a lack of a battery charge. Such an incident can, in turn, introduce unwanted delay in the medical procedure as the technician locates a substitute clipper.

### BRIEF DESCRIPTION OF THE DRAWINGS

The above needs are at least partially met through provision of the clipper described in the following detailed description, particularly when studied in conjunction with the drawings, wherein:

FIG. 1 is a perspective view of a clipper as configured in accordance with various embodiments of these teachings;

FIG. 2 is a front view of the clipper of FIG. 1, as configured in accordance with various embodiments of these teachings;

FIG. 3 is a back view of the clipper of FIG. 1, as configured in accordance with various embodiments of these teachings;

FIG. 4 is a side view of the clipper of FIG. 1, as configured in accordance with various embodiments of these teachings;

FIG. 5 is a detail image of an example actuator that might be found on the clipper of FIG. 1, as configured in accordance with various embodiments of these teachings; and

FIG. 6 is a detail image of a display of the clipper of FIG. 2, as configured in accordance with various embodiments of these teachings.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, pursuant to these various embodiments, an electric clipper is provided including an exterior surface having at least one solar cell integrally included therewith. The solar cell is configured to at least partially charge a battery of the electric clipper. So configured, the electric clipper can be recharged whenever it is in the presence of a light source. By this, even if a clipper is not replaced onto a charging station after use (for example, it may be left on a table or counter), the battery of the electric clipper can be recharged for future uses as long as the solar cell receives sufficient light. Further, because the clipper can utilize light to recharge the battery, the clipper may utilize less power from the charging station and thereby constitute a “greener” charging solution.

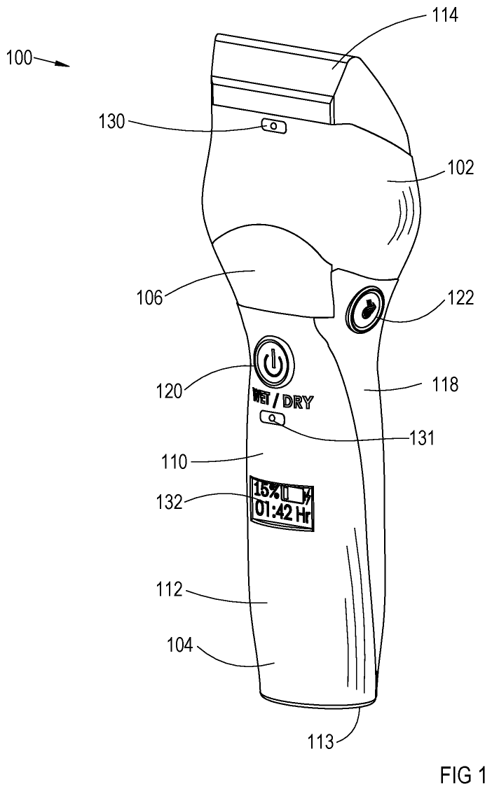

These and other benefits may become clearer upon making a thorough review and study of the following detailed description. Referring now to the drawings, and in particular to FIG. 1, an illustrative clipper compatible with many of these teachings will now be presented.

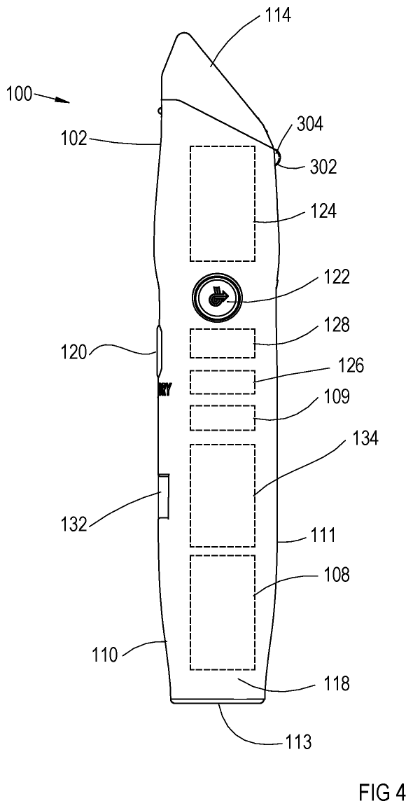

In a first approach, the electric clipper 100 includes a contoured body 102 with an exterior surface 104. The exterior surface 104 has integrally included therewith at least one solar cell 106. The electric clipper 100 also may include one or more batteries 108 (see FIG. 4) such as, for example, rechargeable batteries as are known in the art. The solar cell 106 is configured to at least partially charge the battery 108. Such charging may occur during one or more of the following: while the clipper 100 is in use, while it is not in use, and even while the clipper 100 is also charging via a charging station (such as a charging station that utilizes inductive charging or that provides a charging current via a direct electrical contact. When the battery 108 is being charged by a charging station, the solar cell 106 may contribute additional charge in tandem with the charging station. This may reduce the amount of charge required from a charging station.

Charging by the solar cell 106 may be regulated by one or more battery charging management circuits or modules 109 (see FIG. 4) that manage charging of the battery 108 to ensure that the battery charges at the proper rate, is not over charged, and maintains a proper maximum charge. The battery charging management circuit or module 109 may be incorporated partially or wholly into one or more microprocessors 126, or may be a separate standalone module or circuit 109, as depicted in FIG. 4.

The charging rate offered by the solar cell 106 may vary by the amount or type of available light, the physical size of the solar cell 106, the number of individual solar cells that comprise the solar cell 106, the conversion efficiency of the solar cell 106, as well as other factors. When compared to the charging rate afforded by use of a charging station, the solar cell 106 may provide between 5% and 15% of the rate provided by the charging station. However, the charging rate from the solar cell 106 may be much greater than in other approaches (for example, up to 100% or higher) dependent upon the factors discussed above and the charging rate of the charging station.

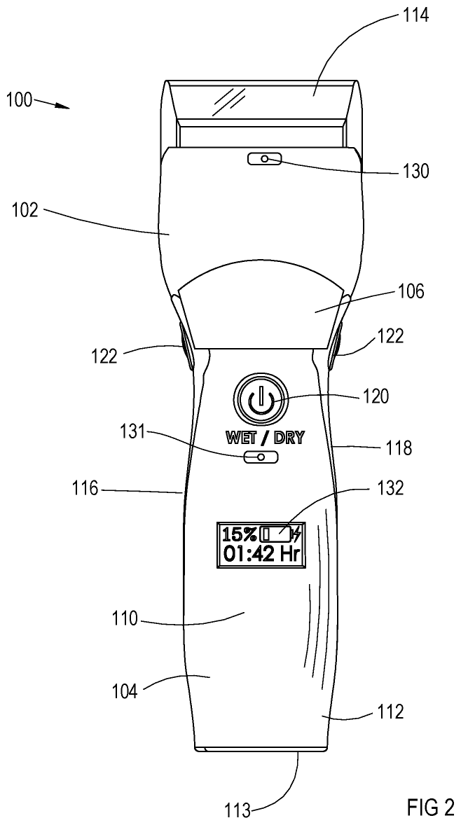

The solar cell 106 (such as a photovoltaic cell) may be a single cell or a plurality of cells forming one or more solar panels. By one approach, the solar cell 106 is integrated with the exterior surface 104 of the clipper body 102 in a single location. FIGS. 1 and 2 illustrate the solar cell 106 integrated with a portion of the exterior surface 104 on the front side 110 of the clipper 100. The front side 110 typically faces upward during normal use, which can allow the solar cell 106 to be exposed to a relatively increased amount of light during use.

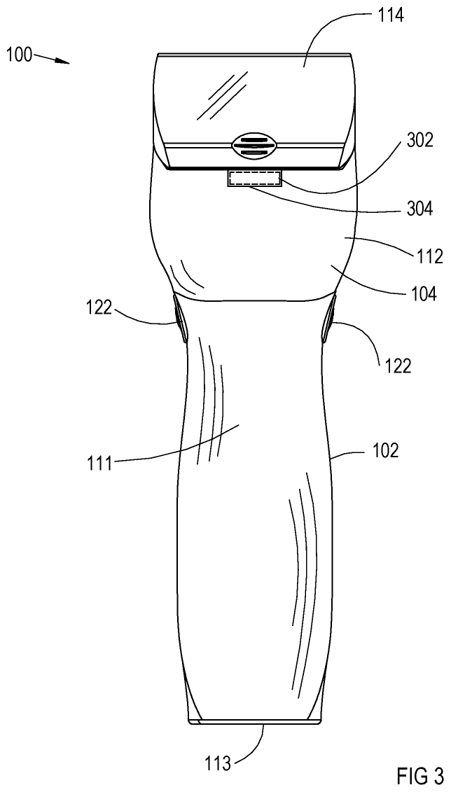

Further, due at least in part to ways in which many users will typically hold the electric clipper 100 during use (for example, like a pen), the electric clipper 100 will at least often be placed on a surface (such as a table or counter) on its back side 111 (shown in FIG. 3) or bottom side 113 (which may be flat to allow the electric clipper 100 to stand upright) with the front side 110 facing upward or outward, again allowing for an increased exposure to light (presuming an ordinary placement of light sources in the application setting).

Other locations on the clipper body 102 may also prove useful for light collection or may suit the particular needs of varying clipper designs. The solar cell 106 may be integrated with a different side of the electric clipper 100 or may span multiple sides. For example, the solar cell 106 may start on the left side 116, stretch across the front 110, and terminate on the right side 118.

As mentioned above, the solar cell 106 may be integrally included with an exterior surface 104 of the contoured clipper body 102. By this, in one example, the solar cell 106 may be directly coupled or connected to the exterior housing 112 of the clipper 100. For example, the exterior housing 112 may include a recess or hole into which the solar cell 106 is fitted and secured by one or more attachment devices (e.g., a screw, nail, tack, bezel, and so forth), connection points molded or formed in the housing 112 and/or solar cell 106 (such as one or more molded push snap or press-fittings), adhesives, sonic or heated welding, or the like.

In another example, the solar cell 106 may be coupled to an internal frame of the clipper 100 (in a similar manner as described above), which frame may be coupled to the exterior housing 112, thus providing integration with the exterior surface 104, albeit indirect. In yet another example, the solar cell 106 is disposed on top of the exterior surface 104, possibly protruding outwardly therefrom. However, an approach that results in a flush fit and/or continuous surface with the exterior surface 104 of the housing 112 may be preferred for many application settings to reduce corners or crevices where foreign matter or microbes may reside.

The solar cell 106 may include a transparent (or substantially transparent) cover that overlies and protects the solar cell 106. By one approach the cover may act as a lens that directs or focuses light onto the solar cell 106. Such a cover may be coupled directly to the solar cell 106 to form a module, or the cover may be separate therefrom. The cover may be coupled to the exterior housing 112 (as described above with respect to coupling the solar cell 106 to the exterior housing 112), or may comprise an integral portion of the exterior housing 112 such as a transparent or translucent window portion thereof.

By other approaches, other additional solar cells (not shown) may be integrated with other sides or surfaces of the exterior surface 104 of the clipper body 102. For example, a second solar cell may be integrated with the front side 110 or with the back side 111 of the electric clipper 100, or one or both of the left and right sides 116, 118 of the electric clipper 100. Each of the multiple solar cells in such an approach may be configured to at least partially charge the battery 108, either together or individually.

Various kinds of batteries 108 may be utilized in the electric clipper 100, such as lithium-ion (Li-ion), nickel-cadmium (NiCd), or nickel-metal hydride (NiMH) batteries. Other common rechargeable battery types may also be utilized. In a preferred embodiment, Li-ion batteries are utilized as Li-ion batteries typically charge quicker and last longer than other rechargeable battery varieties. Further, Li-ion batteries can provide continuous full or near-full power output even as the charge is depleted, thus providing more effective use of the clipper 100 throughout a discharge cycle. In one approach, the batteries 108 are permanently integrated with the clipper 100 so that they are not replaceable. Such an approach affords the ability to permanently seal the clipper body 102 to thereby provide a waterproof result. However, in other approaches, the battery 108 may be replaceable, either by the end user or by a trained service technician.

In some approaches, the electric clipper 100 is a medical clipper (or surgical clipper). Medical clippers are typically used to prepare a surgical site of a patient for surgery prior to a surgical procedure or operation. Hair can inhibit the ability of a surgeon or other personnel from clearly viewing a surgical area (for example, if surgery is to be performed on the scalp). Further, hair can capture bacteria that increase the chance of infection. Thus, removal of the hair at or near the surgical site (entirely or at least to stubble) is essential before performing many surgical procedures or operations.

Medical clippers, such as electric clipper 100, also often utilize disposable one-time-use clipper heads 114 that afford a single use per patient. For example, the electric clipper 100, and more specifically, the clipper body 102, is configured to be mated with a clipper head 114 for use thereon. After use, the clipper head 114 is disposed rather than re-used on other patients to thereby help prevent the spread of diseases and infections. The use of a disposable clipper head 114 serves as at least one point of distinction between medical clippers and grooming clippers (such as typical home-consumer clippers or shavers and clippers or shavers used by professionals such as barbers and hairstylists). Grooming clippers are usually equipped with non-disposable blades suited for a much longer lifespan (and certainly for use with more than a single subject). Often, varying clipper head 114 types are available for use in a medical services application setting, such as, for example, a standard head suitable for most applications, a heavy-duty head for thicker or denser hair, and a small head for delicate and/or more physically-complicated areas. By one approach, the electric clipper 100 can sense the type of clipper head 114 installed thereon and can adjust various performance attributes such as power, speed, and torque to best accommodate that clipper head and/or the anticipated application setting, or possibly adjust power-usage calculations based on the type of clipper head 114. Another point of distinction between medical clippers and grooming clipper may include a medical clipper's ability to be rinsed under flowing water or submerged under water. Other points of distinction between medical and grooming clippers will be readily appreciated by those of skill in the art.

Turning now to FIG. 3, in various embodiments the clipper 100 includes a clipper head release actuator 302 to allow a user to disengage a clipper head 114 from the clipper 100 upon actuation without the user also contacting the clipper head 114. This reduces the possibility of a user becoming contaminated with hair and other foreign matter that may cover the clipper head 114 after use. The clipper head release actuator 302 can comprise, for example, a button that can be actuated or depressed in toward the body 102 of the clipper 100, possibly at an angle corresponding to an angle at which the clipper head 114 is installed, though other actuator types are possible (such as rocker switches, levers, and the like). The actuation action can serve release the clipper head 114 from the clipper body 102, or to both release and slide the clipper head 114 off the clipper body 102.

The actuator 302 may reside in a recess in the external housing 112. Traditionally, when actuated, such an actuator 302 may expose portions of the recess, thus allowing the entry of foreign matter into the recess. Further, even when not actuated, other solutions allow foreign matter to enter the recess. The recess is difficult to clean between uses as it may include various corners and crevices which trap foreign matter. Further, at least portions of the recess may at all times be inaccessible because they are blocked by the actuator 302, even when actuated.

By one approach, the clipper head release actuator 302 of the present disclosure includes a resilient cover 304 that fully covers the clipper head release actuator 302 and prevents foreign matter from entering the body 102 of the clipper 100 (for example, a recess or the inside the external housing 112). The resilient cover 304 may be made of rubber, silicone, or other common resilient materials. Preferably, in its non-actuated state, the resilient cover 304 is made to be flush with or protruding from the external surface 104 of the clipper, though it may protrude into the contoured body 102 by other approaches. When the actuator 302 is actuated, the cover 304 may deform and protrude into the recess, but upon release may then reassume its previous flush or exterior protruding shape. During actuation of the actuator 302, the cover 304 will preferably maintain a seal around the actuator 302, the recess, the exterior housing 112, or a portion of the exterior surface 104 to prevent entry of foreign matter. Such a seal may preferably be water and/or air tight.

Returning now to FIGS. 1 and 2, other actuators or buttons may be provided on the clipper 100. For example, the clipper 100 may include a power actuator 120 or button that toggles operation of the clipper 100 between on and off. The power actuator 120 may also include a resilient cover as discussed above, though other approaches are possible.

In certain approaches, the clipper 100 also includes one or more boost actuators 122 integrated with at least one exterior surface 104 of the electric clipper 100. The boost actuators 122 may also include resilient covers as discussed above, and may be recessed, flush with, or protruding from the external surface 104 of the external housing 112. By one approach, the boost actuators 122 are located on one or both sides 116, 118 of the electric clipper 100 to provide a convenient method of user engagement when the electric clipper 100 is held during use. Because it is presently suggested to hold the clipper 100 like a pen, the placement of the boost actuators 122 keeps them near the thumb and/or index finger to make them easy to actuate during use.

With brief reference to FIG. 5, which illustrates an example actuator 502 that may correspond to the power actuator 120 and/or the boost actuators 122, the actuator 502 may include a raised tactile ring 504 surrounding the actuator 502 to provide tactile feedback as to the location of the actuator 502. The actuator 502 (or resilient cover thereon) may be flat, convex (as shown), or concave. Various symbols 506 may be emblazoned on the actuator 502 indicating the function of the actuator 502.

In use, the boost actuators 122 are configured to effect provision of a boost effect, which includes, in one form, modifying power, speed, or torque provided to the clipper head 114 by a motor 124 (possibly an ultrasonic motor) (see FIG. 4) of the electric clipper 100 in response to being actuated or depressed. In certain approaches, such a modification of the power, speed, or torque provided to the clipper head 114 may comprise increasing or decreasing the power, speed, or torque provided to the clipper head 114. By one approach, a microprocessor 126 or other circuit may detect actuation of the boost actuator 122 and enable the boost effect.

In one approach, the boost effect may remain active only while the boost actuator 122 is actuated. In a different approach, the boost effect is toggled on and off by successive activations of the boost actuator 122. In other approaches, the boost effect is provided for a set amount of time (for example, 1, 2, 5, 10, etc., seconds) upon actuation of the boost actuator 122. Various combinations of these functionalities are possible as well. For example, if the boost effect is provided for a set amount of time, when the boost actuator 122 is actuated again before expiration of that set time, the boost effect may switch off. Alternatively, such an action may reset a timer to provide the set amount of boost effect time again after actuation, even if a boost effect is currently being provided.

By providing the boost effect of additional power, torque, or speed, the clipper 100 is able to accommodate patches or areas of thicker or denser hair without constantly operating in a boosted state, which state depletes battery power quicker. By normally operating in a reduced state of power, torque, or speed, a reduction in overall power usage is realized. However, the boost actuator 122 offers the ability to provide the additional boost effect as needed, possibly to operate the motor 124 at maximum or near maximum output. The boost effect typically utilizes more battery power—but allowing it to be selectively utilized will reduce overall power usage, extend use life between charges, and extend the overall life of the battery 108 (and thus the overall life of the electric clipper 100).

In one approach, an intuitive boost effect is provided. In such an approach, the electric clipper includes a sensing module 128. The sensing module 128 may be implemented in a microprocessor 126 or in other separate circuitry of the electric clipper 100 to detect at least one operating condition of the electric clipper 100 during operation that may correspond to clipper exertion (e.g., how hard the clipper is working). For example, the sensing module may detect the current power, torque, or speed being provided to or exerted on the clipper head 114.

The sensing module 128 may also determine that the operating condition exceeds (e.g., is higher or lower than) an operating condition threshold. In response to exceeding the operating condition threshold, the sensing module 128 may increase or effect an increase in power, torque, or speed. For example, if the sensing module 128 detects that the current speed of the clipper head drops below a certain threshold, it may determine that this relates to a particularly dense patch of hair or a patch of particularly coarse hair and may effect provision of additional power to the clipper head 114 to better accommodate the thicker hair. Similarly, if the sensing module 128 detects that the power being used exceeds a certain threshold, it may responsively effect an increase in the speed of the clipper head 114 to better accommodate the dense/course patch. Other variations are of course possible.

Other aspects of the electric clipper 100 may include at least one illuminator (e.g., illuminator 130 or 131) configured to illuminate an area subject to the clipping operation of the electric clipper 100. For example, one or more light emitting diodes (LED's) or other light sources may be affixed, disposed upon, or incorporated with the external surface 104 of the electric clipper 100 in a single location or in multiple locations. The illuminator(s) 130 or 131 may be aimed generally toward the work area and may be flush with the external surface 104, exist in a shaped recess in the external surface 104 to allow light to shine on the subject area, or exist in a housing that protrudes from the external surface 104 to allow light to shine on the subject area. In one form, an illuminator 130 may be included on the front side 110 slightly below the clipper head 114, as shown in FIGS. 1 and 2, and may be aimed upwardly so that when the clipper head 114 engages the subject area, the work area is illuminated. In another form, an illuminator 131 may be provided instead of or in addition to illuminator 130. Illuminator 131 may be located further down on the clipper body 102, possibly below a power actuator 120. This position may offer a wider beam of light on a work area as it is further away from the work area when the clipper head 114 engages the subject area. As mentioned elsewhere, it is recommended to hold the clipper 100 like a pen during use (e.g., by gripping the sides 116, 118 with the index finger and thumb, the palm generally facing the clipper head 114, while the back side 111 of the clipper rests in the thenar space of the hand, being the space between the thumb and index finger). By holding the clipper in this manner, illuminator 131 is not occluded by the hand (as might occur if held in an over-hand manner, with the palm facing away from the clipper head 114 and contacting the front side 110). Further, in some approaches, the placement of illuminator 131 may encourage using the recommended pen grip as an over-hand grip may occlude the illuminator 131. Other locations for placement of the illuminator(s) 130 or 131 may also be suitable to illuminate the area subject to the clipping operation such as, for example, on the clipper head 114, on the sides 116, 118, or other suitable locations. The illuminator(s) 130 or 131 may be configured to only be switched on while the electric clipper 100 is on (or possibly also for an additional short amount of time after the clipper is turned off). Alternatively, a separate and possibly dedicated actuator (such as a switch or button) may be provided to control this illumination functionality. Alternatively still, the illuminator(s) 130 or 131 may be activated by sensing that the electric clipper 100 is being held, for example, by sensing its own movement or through contact sensors configured to detect a user's hand.

The electric clipper 100 may include one or more displays 132 in some approaches. Such a display 132 may be integrated into the exterior surface 104 of the exterior housing 112 of the electric clipper. The display 132 may include a transparent cover that is generally flush with the exterior surface 104 to prevent build up of foreign matter or bacteria. The display 132 may be a liquid crystal display (LCD), a display formed from a plurality of light-emitting diodes, or the like, and may be backlit. The backlight may be activated/deactivated upon turning the electric clipper 100 on or off, activating a boost actuator 122, being coupled to or decoupled from a charging power source, sensing that the electric clipper 100 is being held, or by actuating or depressing a dedicated actuator or button, to note but a few options in these regards.

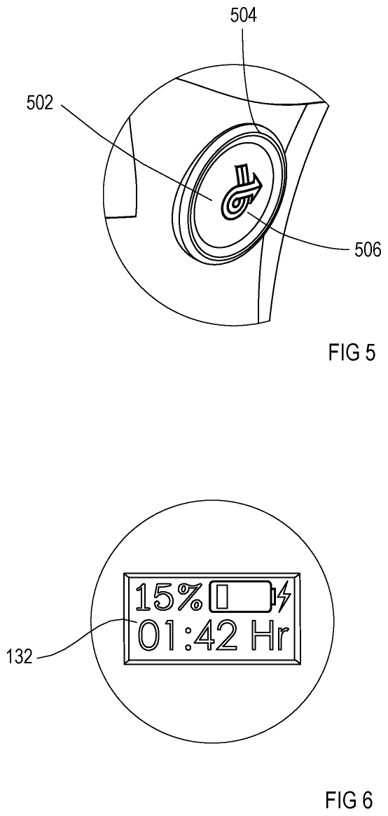

Turning briefly to FIG. 6, the display 132 may be configured to show the amount of power remaining on the current charge in one or more of a relative percentage indication, a time-remaining metric, or a graphical bar of power, though other depictions are contemplated as well. Such an approach reduces the chance that the battery 108 for the electric clipper 100 becomes entirely depleted prior to or during a successive use of the electric clipper 100 as the display 132 can indicate to a user that the clipper needs (or likely needs) to be recharged.

The display 132 may indicate other information as desired, such as whether the electric clipper 100 is currently being charged, activation status of a boost effect, total or average usage (in time), or charging efficiency breakdown (that is, what percentage of the battery charge is or was from solar power as versus a charging station).

Further, the electric clipper 100 may include various externally-exposed portions that are made of or include anti-microbial materials. Particularly, a majority of the plurality of exterior surfaces of the electric clipper 100 may include an anti-microbial material or a material that exhibits anti-microbial properties. These surfaces may include the exterior surface 104 of the clipper body 102, the clipper head 114, the clipper head release actuator 302 and/or cover 304, other actuators and/or covers (e.g., the power actuator 120 or boost actuators 122), and a display 132 or display cover. These surfaces may be impregnated with anti-microbial materials or may themselves comprise anti-microbial materials such as, for example, copper.

In one approach, the electric clipper 100 may include a vacuum 134 (see FIG. 4) configured to suction foreign matter during operation of the electric clipper 100 and/or to store foreign matter during operation of the electric clipper 100. The vacuum 134 may be a fan or turbine driven system, a vacuum pump, or another suction system. Operation of the vacuum 134 may be directly coupled to operation of the electric clipper 100, or may be separately activated through a separate actuator. The vacuum 134 may include a canister or bin that collects and stores foreign matter such as hair, liquid, and microbes during operation, which canister or bin can be emptied after use to ensure the clipper 100 is suitable for a succeeding use. Vacuums are currently utilized in various grooming clippers, specifically, consumer-grade clippers and shavers (as distinct from medical clippers). Accordingly, implementation of a vacuum into a consumer-grade clipper or shaver is understood. Though not previously contemplated, many details regarding implementation of a vacuum into a medical clipper may parallel details relating to implementation in the consumer-grade counterparts. Such details are understood in the art and are omitted here for the sake of brevity.

By at least one approach, the exterior surface 104 of the electric clipper 100 may be constructed of carbon fiber, plastic, resin, metal, glass, or another suitable material or combination of materials. For example, the exterior surface 104 may be made of a single molded plastic housing 112 or constructed of multiple molded plastic portions coupled together to form the exterior housing 112. Such coupling may be achieved through one or more attachment devices (e.g., screws, nails, tacks, or the like), connection points molded or formed in the housing portions (such as one or more molded push snaps or press-fittings), adhesives, sonic or heated welding, or the like.

In one example, the exterior surface 104 may include various trade dress aspects such as a carbon fiber appearance and a logo emblazoned (possibly with a mirror appearance) in a conspicuous location, such as above the power actuator 120 and solar cell 106. Different materials and/or appearances may be utilized for different locations on the exterior surface 104. For example, while the majority of the exterior surface 104 may include a carbon fiber appearance, portions of the back side 111 may utilize a material of different color and/or texture. Such an approach may aid in gripping the electric clipper 100 (when held like a pen) to avoid slippage and improve comfort.

Preferably, the electric clipper 100 as a whole is waterproof to allow for it to be regularly cleaned under running water or by submersion between uses. In one approach, the clipper 100 conforms to a liquid ingress protection rating of level 7 (IPX7), which provides ingress protection against immersion up to 1 meter for at least 30 minutes. However, other lesser or greater waterproofing ratings may be utilized where appropriate.

One or more methods corresponding to the above described electric clipper 100 are also contemplated. In one approach, a method includes providing at least some charge to the battery 108 of the electric clipper 100 from at least one solar cell 106 integrally coupled to at least one exterior surface 104 of the electric clipper 100. This may be achieved, for example, by a user exposing the electric clipper 100, and more particularly the solar panel 106 of the electric clipper 100, to a light source. By other approaches, the method also may include various ones of the following: temporarily mating a disposable one-time-use clipper head 114 to the electric clipper 100 in advance of using that one-time-use clipper head 114 for only a single subject and then removing (and disposing of) the one-time-use clipper head 114 following use of that head 114 for the single subject; providing anti-microbial properties to at least a majority of a plurality of exterior surfaces 104 of the electric clipper 100; providing illumination via the electric clipper 100 by one or more illuminators 130, 131 to an area subject to a clipping operation of the electric clipper 100; or employing a vacuum 134 coupled to the electric clipper 100 to suction foreign matter during a clipping operation and/or storing foreign matter during such an operation.

In varying approaches, the method may also include (in lieu of the foregoing or in combination therewith) detecting actuation of at least one boost actuator 122 integrally coupled to at least one exterior surface 104 of the electric clipper 100; and effecting modification of power, torque, and/or speed provided to a clipper head 114 by a motor 124 in response to detecting the actuation of the boost actuator 122. Effecting the modification may comprise, for example, effecting an increase in power, torque, and/or speed provided to the clipper head 114. The method might also include detecting at least one operating condition of the electric clipper 100 during operation corresponding to clipper exertion; determining that the at least one operating condition exceeds an operating condition threshold; and responsively modifying (for example, by increasing) at least one of power, torque, and speed being currently provided to a clipper head 114.

The method may also include disengaging a clipper head 114 from the electric clipper 100 without a user contacting the clipper head 114 in response to actuation of a clipper head release actuator 302 of the electric clipper 100, whereby foreign matter is prevented from entering a body 102 of the electric clipper 100 when the clipper head 114 release actuator 302 is actuated. In one approach, this may further include preventing foreign matter from entering the body 102 by a resilient cover 304 covering the clipper head release actuator 302.

In another approach, a method may include preparing a surgical site of a patient (perhaps by a medical professional) by removing hair located at or near the surgical site with an electric clipper 100. In this approach, the electric clipper 100 includes at least one exterior surface 104 having at least one solar cell 106 integrally included therewith, which solar cell 106 is configured to at least partially charge a battery 108 of the electric clipper 100. The method may further include performing at least one surgical procedure on the patient at the surgical site after preparing the surgical site.

So configured, an electric clipper 100 is provided that can be recharged whenever it is in the presence of a suitable light source. By this, even if a clipper 100 is not replaced onto a charging station after use, its battery 108 can still nevertheless be recharged for future uses as long as the solar cell 106 receives light. Further, because the clipper 100 can utilize light to recharge the battery 108, the clipper 100 can be configured to utilize less power from the charging station to recharge the battery 108, resulting in a more “green” charging solution.

Other apparent benefits are derived from various other elements and aspects described herein. For example, a boost effect can be provided to allow a more economical power usage during normal operation while simultaneously providing additional power, torque, or speed when needed. Further, foreign matter and microbes are prevented from entering the clipper body 102 when a clipper head release actuator 302 or other actuators (power actuator 120 or boost actuator 122) are actuated by use of resilient covers. Anti-microbial properties of an exterior surface 104 prevent spreading of microbes and disease.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

What is claimed is:

1. A method comprising:
providing an electric clipper, wherein the electric clipper comprises:
a body having an exterior surface;
at least one battery disposed within the body;
a motor disposed within the body;
a clipper head, operably coupled to the body, that is powered by the motor and that is configured to clip hair;
a power actuator integrated with the exterior surface of the body that is configured to selectively connect and disconnect the at least one battery and the motor to thereby power on and off the motor in response to corresponding actuations by a user such that when the motor is powered on by the power actuator the motor operates at a normal level of performance;
at least one boost actuator integrated with the exterior surface of the body and configured to provide increased operating performance of the clipper head when actuated by a user; and
a vacuum at least partially disposed within the body and being selectively connectable and disconnectable from the battery.

2. The method of claim 1 wherein the body of the electric clipper has at least two opposing sides and wherein the at least one boost actuator is located on a first one of the two opposing sides.

3. The method of claim 2 wherein another of the at least one boost actuator is located on a second one of the two opposing sides, such that both opposing sides of the body have a corresponding boost actuator that each independently, when actuated by a user, provide increased operating performance of the clipper head.

4. The method of claim 1 wherein the vacuum comprises a fan driven system.

5. The method of claim 1 wherein the vacuum comprises a turbine driven system.

6. The method of claim 1 wherein the vacuum comprises a vacuum pump.

7. The method of claim 1 wherein operation of the vacuum is directly coupled to operation of the electric clipper.

8. The method of claim 1 further comprising a separate actuator that controls actuation of the vacuum.

9. The method of claim 1 wherein the vacuum comprises a bin configured to store foreign matter during operation of the vacuum.

10. An electric clipper comprising:
a body having an exterior surface;
at least one battery disposed within the body;
a motor disposed within the body;
a clipper head, operably coupled to the body, that is powered by the motor and that is configured to clip hair;
a power actuator integrated with the exterior surface of the body that is configured to selectively connect and disconnect the at least one battery and the motor to thereby power on and off the motor in response to corresponding actuations by a user such that when the motor is powered on by the power actuator the motor operates at a normal level of performance;
at least one boost actuator integrated with the exterior surface of the body and configured to provide increased operating performance of the clipper head when actuated by a user; and
a vacuum at least partially disposed within the body and being selectively connectable and disconnectable from the battery.

11. The electric clipper of claim 10 wherein the body of the electric clipper has at least two opposing sides and wherein the at least one boost actuator is located on a first one of the two opposing sides.

12. The electric clipper of claim 11 wherein another of the at least one boost actuator is located on a second one of the two opposing sides, such that both opposing sides of the body have a corresponding boost actuator that each independently, when actuated by a user, provide increased operating performance of the clipper head.

13. The electric clipper of claim 10 wherein the vacuum comprises a fan driven system.

14. The electric clipper of claim 10 wherein the vacuum comprises a turbine driven system.

15. The electric clipper of claim 10 wherein the vacuum comprises a vacuum pump.

16. The electric clipper of claim 10 wherein operation of the vacuum is directly coupled to operation of the electric clipper.

17. The electric clipper of claim 10 further comprising a separate actuator that controls actuation of the vacuum.

18. The electric clipper of claim 10 wherein the vacuum comprises a bin configured to store foreign matter during operation of the vacuum.
